(19) United States
(12) Patent Application Publication
(10) Pub. No.: US 2004/0097925 A1
(43) Pub. Date: May 20, 2004

(54) Cervical spine stabilizing system and method

(76) Inventors: Frank H. Boehm JR., Utica, NY (US); Benedetta D. Melnick, Rome, NY (US)

Correspondence Address: Paul J. Farrell, Esq., DILWORTH & BARRESE, LLP, 333 Earle Ovington Blvd., Uniondale, NY, 11553, US

(21) Appl. No.: 10/457,237

(22) Filed: Jun. 9, 2003

Related U.S. Application Data
(60) Provisional application No. 60/387,141, filed on Jun. 7, 2002.

(51) Int. Cl. A61B 17/56

(52) U.S. Cl. 606/61

(57) ABSTRACT

A spine stabilizing system for retaining a spinal column in a desired spatial relationship during diskectomy and fusion procedures has a plate configured to be positively centered along the midline of the spinal column to maintain the adjacent vertebrae of the spinal column in the desired spatial relationship until the fusion is completed.

### PRIORITY

This application claims priority to provisional application entitled “CERVICAL SPINE STABILIZING SYSTEM AND METHOD” filed in the United States Patent and Trademark Office on Jun. 7, 2002 and assigned Serial No. 60/387,141, the contents are hereby incorporated by reference.

### BACKGROUND OF THE INVENTION

1. Field of the Invention

The present invention relates to a stabilizing apparatus and method for retaining vertebrae of a spinal column in a desired spatial relationship, and in particular, to a plate and instrumentation for stabilizing the cervical spine during a diskectomy and fusion procedure.

2. Discussion of the Related Art

There are many known devices and procedures for retaining vertebrae of the spine in a desired spatial relationship during diskectomies and fusion procedures. These devices maintain the vertebrae in fixed relation with respect to each other by securing the device into the bone of the vertebrae, typically by means of screws. Once secured, the device stays in place during the diskectomy and subsequent fusion procedure. During the procedure, after an incision is made, the device is placed on the spine and is secured to the vertebrae above and below the disk, which is to be removed. The surgeon aligns the device, usually by “eyeballing” the device with respect to the midline of the spine, drills holes into the bones of the vertebra, and then places the screws into the bone through holes in the device.

The devices and methods of the prior art suffer in general the disadvantage in that proper positioning and true alignment can only be achieved to the degree of accuracy with which the surgeon places the device on the spine. Once the holes are drilled, the patient may be left with a device that is not in true alignment. In the confined region of the cervical spine, a misaligned device can cause serious injury to the patient, particularly after the fusion procedure since the device is left in the patient to ensure fusion occurs. Furthermore, re-drilling the bones of the vertebra can weaken the strength of the bones, since the bones of the cervical spine are smaller than the rest of the spine and may not accommodate multiple holes being drilled in such a small area.

The devices of the prior art also tend to be cumbersome and are more suited to the thoracic or lumbar spine. Many include rods that permit adjustment of the spatial relationship between the top portion of the device and the bottom portion, so that it can be used to span different vertebrae in the spine. However, these rods are of limited use in the confined region of the cervical spine.

Therefore, a need exists for a low profile plate device for stabilizing the vertebrae of the spine, in particular the cervical spine, which is adjustable in length to the permit its use with different body sizes. A need also exists for a method of implanting a spine stabilizing plate which allows for accurate placement of the plate with respect to the midline of the spine and prevents damage to the bone of the vertebrae in the event a misalignment occurs and the procedure must be redone.

### SUMMARY OF THE INVENTION

It is an object of the present invention to provide a spine stabilization system and instrumentation that facilitates accurate placement of the respect to the midline of the spine.

It is also an object of the present invention to provide a spine stabilization system and instrumentation that is particularly suited for use with the cervical spine.

It is a further object of the present invention to provide a low profile cervical stabilization plate that is adjustable in length.

It is yet a further object of the present invention to provide a method for accurately placing a cervical stabilization plate with respect to the midline of the spine.

A still further object of the present invention is to provide a method for facilitating a diskectomy and fusion procedure utilizing a stabilizing plate and associated instrumentation that ensures accurate alignment of the plate with respect to the midline of the spine and which minimizes damage to the bones of the vertebra during the alignment of the plate of the vertebra.

The above and other objects are achieved by the cervical spine stabilization method and system of the present invention. The system and instrumentation of the invention comprises at least a cervical stabilizing plate, a temporary screw placement guide and a locator screw. The cervical stabilization plate includes a frame having an upper portion, a lower portion and at least one intermediate portion extended between the upper and lower portions. Each upper and lower portion of the frame includes a central screw hole for facilitating positioning of the plate onto the vertebrae of the spinal column, and at least one lateral screw hole for mounting the plate to the vertebrae by a bone screw.

In a preferred embodiment, the cervical stabilization plate is formed by a one-piece frame. In another embodiment, the plate is comprised of a pair of generally U-shaped frame members that face each other when the frames are assembled. The legs of each of the members are joined to each other through the provision of a ratchet-type mechanism that allows for adjustment of the distance between the upper and lower portions of the frame, so that the device can be utilized on different body sizes. Preferably the width of the upper portion is less than the width of the lower portion, which takes into account the narrowing of the width in the cervical spine as the spine moves toward the base of the skull. In addition, a bridge may be provided between the legs of one or both of the U-shaped members to provide additional support.

A method for facilitating a diskectomy and fusion procedure is also provided. In the method of the present invention, after an incision is made, the locator screw of the present invention is placed in the disc to be removed and aligned by the surgeon. A temporary-screw placement guide is placed over the locator screw, and the vertebra are marked through the holes of the placement guide. Also, temporary screws may be placed into the vertebrae above and below the disc. After vertebra are marked, and/or the temporary screws are in place, alignment is checked through an imaging technique, such as fluoroscopy. If alignment is not confirmed when the temporary screws are in place, the temporary screws are removed, re-aligned, and the procedure continues. If the alignment is confirmed, the bones are drilled through the bone screw holes in the upper and lower portions of stabilizing plate. Permanent screws are then inserted and screwed into the bones. Alignment is then checked again through fluoroscopy, and if alignment is confirmed, then the disk is removed and the fusion procedure is performed.

### BRIEF DESCRIPTION OF THE DRAWINGS

The above objects, features and advantages of the present invention will become more readily apparent and understood with references to the following detailed description of preferred embodiments of the invention, taken in conjunction with the following drawings, in which:

FIG. 1 is perspective view of the cervical plate of the present invention in place on the vertebrae of the spine;

FIG. 2 is a partial cross-sectional view of the plate of FIG. 1, taken along the line 22 of FIG. 1;

FIG. 3 is a plan view of a second embodiment of the plate of the present invention;

FIG. 4 is a plan view of a third embodiment of the plate of the present invention;

FIG. 5 is a plan view of a fourth embodiment of the plate of the present invention;

FIG. 6 is an exploded perspective view of the plate of FIG. 5;

FIG. 7 is a plan view of a fifth embodiment of the plate of the present invention;

FIG. 8 is a partial cross-sectional view illustrating placement of a locator screw into the disc that is to be later removed;

FIG. 9 is a perspective view illustrated positioning of a temporary screw placement guide of the present invention onto the locator screw;

FIG. 10a is a perspective view illustrating a first drill guide; FIG. 10b is a perspective view illustrating a second drill guide; and FIG. 10c is a top plan view of the drill guide of FIG. 10b; and

FIG. 11 is a flowchart illustrating the method of facilitating a diskectomy and fusion procedure of the present invention.

### DETAILED DESCRIPTION

The present invention relates to an apparatus and method for retaining vertebrae of a spinal column in a desired spatial relationship. The present invention is illustrated in association with cervical vertebrae of a human spinal column. It should be understood that vertebrae other than cervical vertebrae of a human spinal column may be retained with the apparatus and method of the present invention.

Preferred embodiments of the presently disclosed cervical spine stabilization device and method of using the same will now be described in detail with reference to the drawings in which like reference numerals designate identical or corresponding elements in each of the several views.

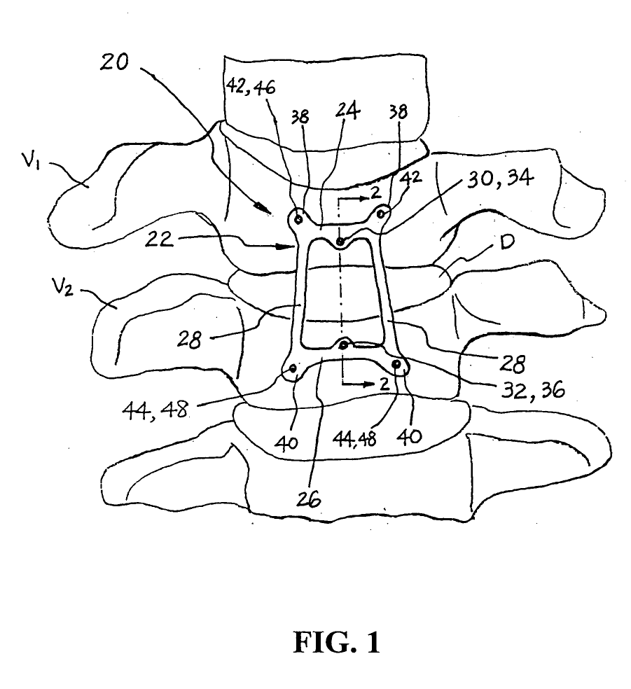

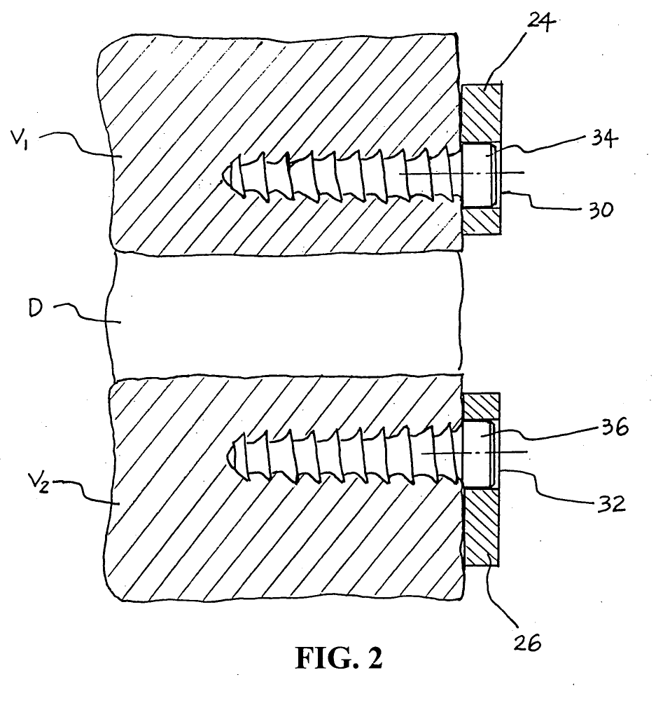

Referring to FIGS. 1-2, the cervical stabilization plate of the present invention is shown in place on the cervical vertebrae “V₁” and “V₂” located above and below the disc to be later treated or removed. Plate 20 includes a substantially square shaped frame 22. Frame 22 includes upper portion 24, lower portion 26 and at least one intermediate portion 28 extended between the upper and the lower portions. Upper and lower portions respectively include central screw holes 30, 32 at the center positions thereof for receiving temporary positioning screws 34 and 36, which will be later described in detail. Upper and lower portions preferably include ear portions 38, 40 and at least one lateral screw hole 42, 44, preferably four holes at the four corners of the frame. Lateral screw holes 42, 44 are provided for mounting the plate 20 to the vertebrae by bone screw 46, 48, which will be described later in detail.

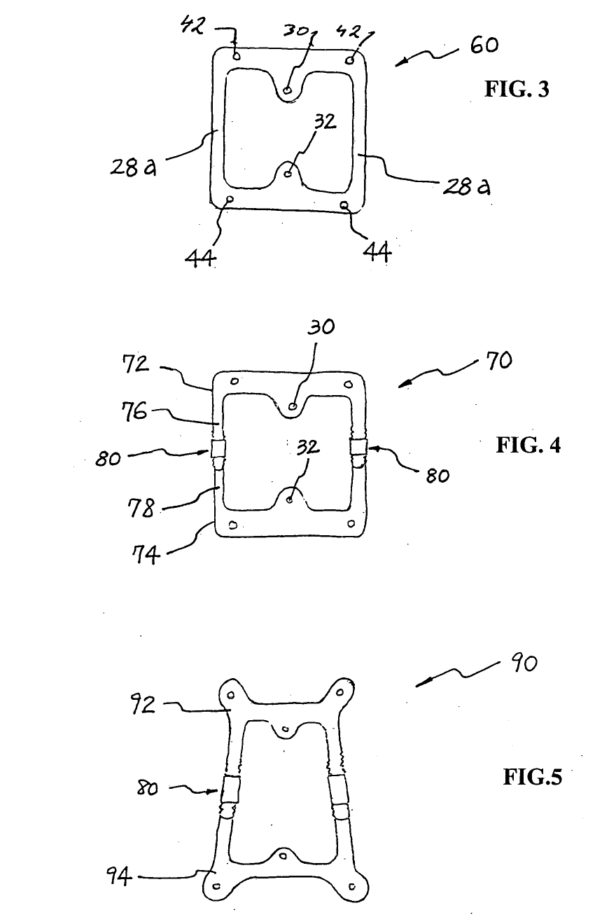

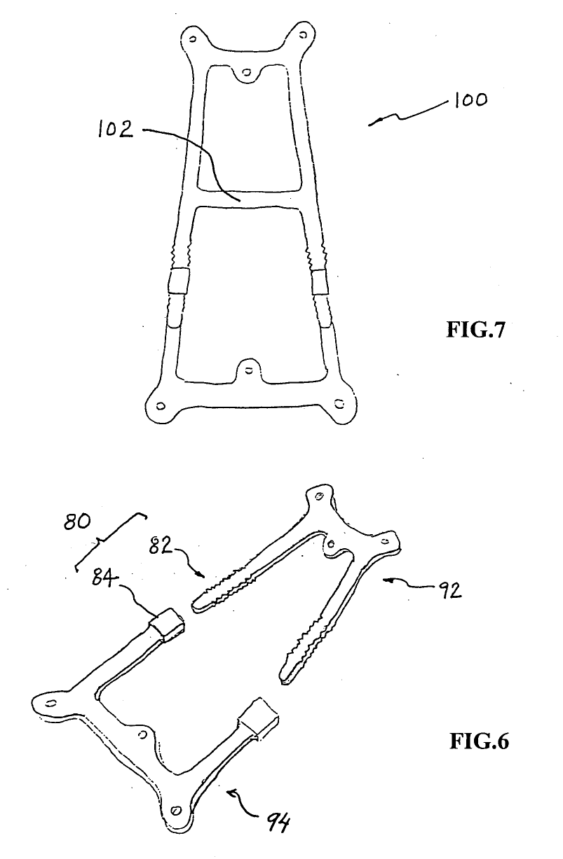

Referring now to FIGS. 3-7, further embodiments of the cervical stabilization plate of the present invention are shown. Cervical stabilization plate 60 (FIG. 3) is similar to the plate 20 of FIG. 1 except that plate 60 is of square configuration with two parallel intermediate leg portions 28a. Cervical stabilization plate 70 (FIG. 4) is similar to the plate 60 of FIG. 3 except that plate 70 consists of a pair of generally U-shaped frame members 72, 74 that face each other when the frames are assembled as shown in the figure. Frames 72, 74 included leg portions 76, 78 which are joined to each other by ratchet mechanism 80. As shown in FIG. 6, ratchet mechanism 80 includes ratchet 82 at one leg portion and connection member 84 at the other leg portion to be joined. Ratchet mechanisms are known in the art and allow for adjustment of the distance between the two joining frames, so that the plate 70 can be utilized on different body sizes or providing fine adjustment at the site. Cervical stabilization plate 90 (FIGS. 5-6) is similar to the plate 20 of FIG. 1 except that plate 90 is formed by a pair of generally U-shaped frame members 92, 94 and joined by ratchet mechanism 80 as described above. Cervical stabilization plate 100 (FIG. 7) is similar to the plate 90 of FIG. 5 except that plate 100 further includes bridge 102 extending between two leg portions for providing additional support to the plate.

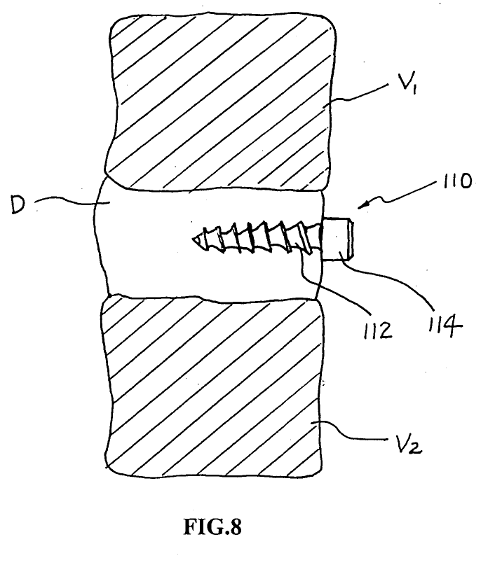

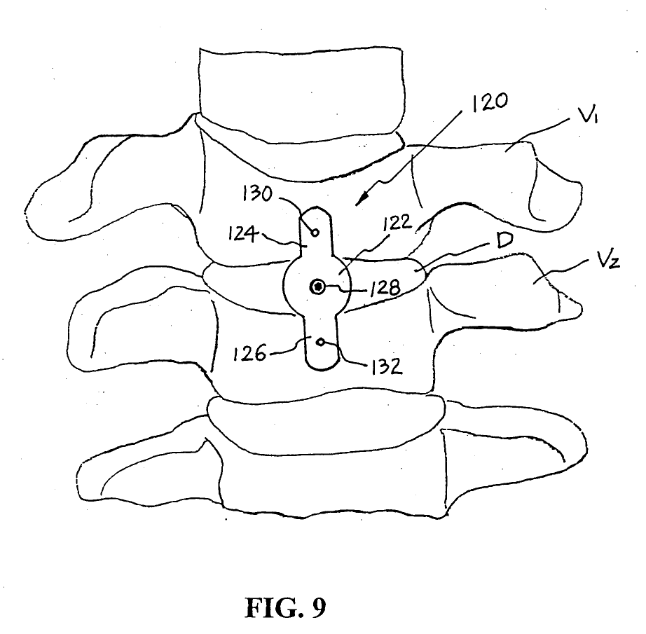

Referring now to FIGS. 8-9, further instrumentation for stabilizing vertebrae in accordance with the present invention is described herein. Locator screw 110 includes screw portion 112 and head portion 114 preferably with a circular configuration. Temporary screw placement guide 120 includes body portion 122, upper portion 124 and lower portion 126. Body portion 122 includes locator hole 128 for receiving the head portion 114 of the locator screw 110 in a slidable fit manner. Upper and lower potions 124, 126 include temporary screw holes 130, 132 for guiding the insertion of temporary positioning screws 34, 36 at suitable locations into the vertebrae V₁ and V₂. Temporary positioning screws 34, 36 have a similar configuration to locator screw 110 described above.

Referring to FIGS. 8-11, a method for stabilizing vertebrae utilizing the system of the present invention is described herein.

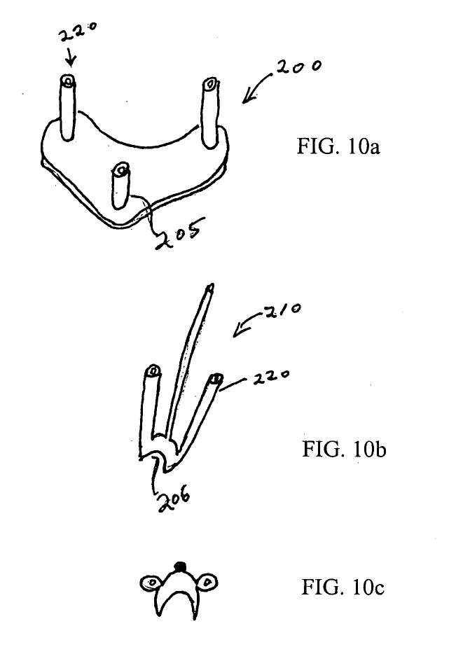

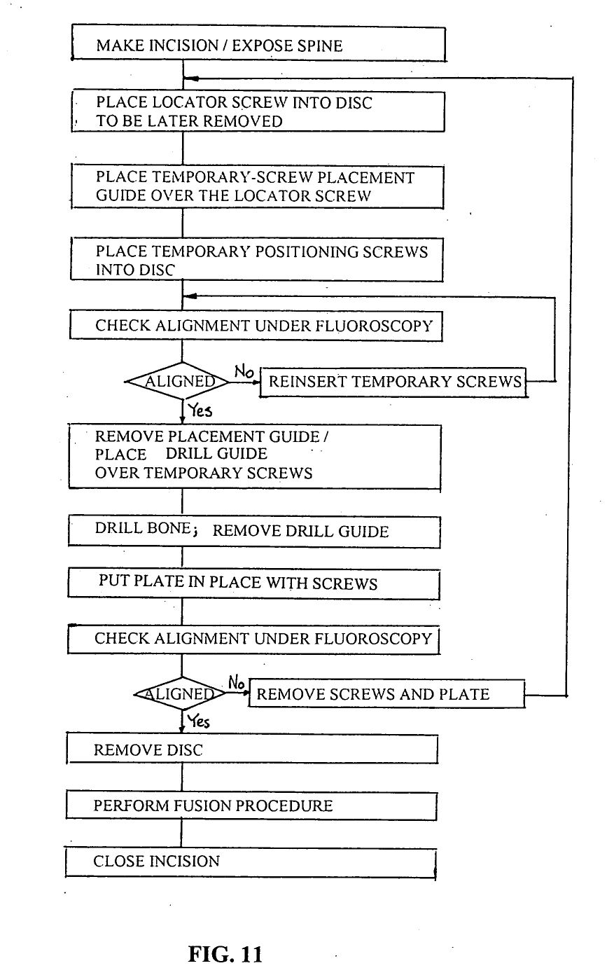

The surgical operations site involving cervical vertebrae to be treated is accessed through incision by a surgical knife and thereby exposing the target spinal area. Locator screw 110 (FIG. 8) is placed by an insertion tool into disc “D” at a midline location. The location of the screw 110 may be accurately checked through an imaging technique, such as fluoroscopy. Temporary screw placement guide 120 (FIG. 9) is then placed over the locator screw 110 with the head of the screw 110 inserted over locator hole 128 of the guide 120. The location of temporary screw holes is adjusted to coincide the midline of the spine by rotating the guide 120 about the locator screw 110. Temporary positioning screws 34, 36 (FIG. 2) are then placed through the screw holes 130, 132 of the guide 120 and into the vertebrae V₁ and V₂. It is preferable to drill pilot holes into the vertebrae V₁ and V₂ before the insertion of the temporary screws 34, 36 as described above, although marking of the vertebra is also contemplated. Alignment of the temporary screws with respect to the spine is checked through an imaging technique such as fluoroscopy. If alignment is confirmed, guide 120 is then removed, and a drill guide 200, 210 such as shown in FIGS. 10a-c is placed over temporary screws 34,36 at post 205 or notch 206. Holes are drilled through the guide posts 220. The drill guide is removed after confirming alignment, and the cervical stabilization plate of the present invention, such as plates 20, 60, 70, 90 and 100 in FIGS. 1-7, is placed over the temporary screws 34 and 36 with such screws fit into the screw holes 30, 32 as shown in FIG. 2. If alignment is not confirmed, the temporary screws are removed and the aforementioned procedure is repeated. Bone screws 46, 48 are inserted through the screw holes 42, 44 of the plate, thereby securing the plate firmly to the cervical spine. Alignment of the plate may be again checked using the fluoroscopy procedure. If alignment is confirmed, disc “D” is then removed and the subsequent fusion procedure is performed. FIG. 11 summarizes the process of the present invention.

It will be understood that various modifications may be made to the embodiments disclosed herein.

What is claimed is:

1. A spine stabilizing system for retaining a spinal column in a desired spatial relationship during diskectomy and fusion procedures, comprising a plate extending along an axis and between at least two adjacent vertebrae of the spinal column and operative to have the axis thereof aligned with a midline of the spinal column before attachment of the plate to the at least two adjacent vertebrae at locations spaced laterally from the midline.

2. The spine stabilizing system of claim 1, wherein the plate comprises
a frame provided with at least two holes formed along the axis in a spaced relationship,
the at least two holes each being configured to receive a respective temporary positioning fastener penetrating the spinal column upon alignment of the axis and the midline to temporarily stabilize the spinal column in the desired spatial relationship.

3. The spine stabilizing system of claim 2, wherein the frame comprises
a plurality of apertures spaced laterally from the axis, and
positioning fasteners each configured to extend through a respective aperture to attach the plate to the at least two adjacent vertebrae at the locations so as to maintain the desired spatial relationship of the spinal column during diskectomy and fusion procedures.

4. The spine stabilizing system of claim 3, wherein the frame is centered along the axis thereof and is configured to have
a pair of axially spaced sides, each extending transversely to the axis and having a respective one of the at least two holes and a pair of the plurality of apertures spaced laterally in opposite directions from the respective hole, and
a pair of spaced portions extending between and coupled to the two axially spaced sides to form the frame.

5. The spine stabilizing system of claim 3, wherein the frame is monolithic.

6. The spine stabilizing system of claim 4, wherein the frame includes a plurality of segments operative to selectively engage one another to adjust an axial length of the frame to correspond to the desired spatial relationship of the spinal column.

7. The spine stabilizing system of claim 6, wherein the plurality of segments each has a generally U-shaped cross section including a respective one of the two axially spaced side and the two spaced portions, the spine stabilizing system further comprising a plurality of couplers configured to receive free ends of the two spaced portions of the frame and to fix the plurality of segments relative to one another upon establishing the desired spatial relationship of the spinal column.

8. The spine stabilizing system of claim 7, wherein each of the couplers includes a respective ratchet mechanism.

9. The spine stabilizing system of claim 4, wherein each of the axially spaced sides spaced sides of the frame is provided with
a respective eyelet, each of the eyelets being configured to have a respective one of the two axially spaced holes, and
a pair of ear portions each having a respective one of the plurality of apertures.

10. The spine stabilizing system of claim 4, wherein the frame further includes an intermediary side extending generally parallel to the axially spaced sides and between the pair of spaced portions to reinforce the frame against compressive loads experienced by the spinal column.

11. The spine stabilizing system of claim 4, wherein the frame has a polygonal shape, and wherein the spaced portions extend parallel to or non-parallel to one another.

12. A spine stabilizing system for retaining a spinal column in a desired spatial relationship during diskectomy and fusion procedures, comprising:
a temporary fastener placement guide configured to provide first locations for at least two holes spaced apart along a midline of the spinal column at a distance spanning at least adjacent vertebrae of the spinal column;
a plurality of temporary positioning fasteners each received in a respective one of the at least two holes, the temporary fastener placement guide being configured to detach from the plurality of temporary positioning fasteners upon installation thereof; and
a plate extending along an axis and mountable over the plurality of temporary positioning screws so that the axis coincides with the midline of the spinal column before the plate is attached to the at least two adjacent vertebrae at second locations spaced laterally from the midline to provide a desired spatial relationship of the spinal column during subsequent diskectomy and fusion procedures.

13. The spine stabilizing system of claim 12, wherein the temporary fastener placement guide has at least one intermediary portion juxtaposed with an intervertebral disc and provided with a locator slot, which is traversed by a screw driven into the intervertebral disc so that the temporary fastener placement guide is rotatable about the screw in a desirable position wherein the first locations are positioned along the midline of the spinal column.

14. The spine stabilizing system of claim 12, further comprising a drill guide having a plurality of posts arranged to be aligned with the first and second locations and configured to receive a drill for forming apertures at the second locations.

15. The drill guide of claim 14, wherein one of the plurality of posts is configured to selectively engage the plurality of temporary fasteners to allow for further alignment of each of the remaining posts with a respective one of the second locations.

16. The drill guide of claim 14, wherein the drill guide has a notch configured to selectively engage the plurality of temporary fasteners to have the plurality of posts aligned with the second locations.

17. A method for retaining at least two adjacent vertebrae of a spinal column in a desired spatial relationship during diskectomy and fusion procedures, comprising the steps of:
removably attaching a central portion of a temporary fastener placement guide along a midline of the spinal column to an intervertebral disc located between adjacent vertebrae;
rotating the temporary fastener placement guide about the central portion thereof to mark spaced locations located along the midline of the spinal column on each of the adjacent vertebrae;
driving a plurality of temporary fasteners each into a respective one of the adjacent vertebrae at a respective one of the marked spaced locations;
placing a plate having a central axis over the plurality of temporary fasteners, thereby centering the plate on the adjacent vertebrae along the midline; and
fastening opposite sides of the plate spaced laterally from the central axis to the adjacent vertebra to maintain a desired spatial relationship between the adjacent vertebrae during diskectomy and fusion procedures while having the plate centered along the midline of the spinal column.

18. The method of claim 17, further comprising fluroscopically verifying alignment of the plate after driving the plurality of temporary fasteners.

19. The method of claim 17, further comprising fluroscopically verifying alignment of the plate after fastening the opposite sides of the plate to the adjacent vertebrae.

20. The method of claim 17, further comprising removing the fastener placement guide upon attaching the plurality of temporary fasteners.
